(19) United States
(12) Patent Application Publication
(10) Pub. No.: US 2015/0335293 A1
(43) Pub. Date: Nov. 26, 2015

(54) SYSTEMS AND TECHNIQUES TO DETERMINE WHETHER A SIGNAL IS ASSOCIATED WITH A PERIODIC BIOLOGIC FUNCTION

(71) Applicant: Maxim Integrated Products, Inc., San Jose, CA (US)

(72) Inventors: Daniel S. Christman, Campbell, CA (US); Arpit Mehta, Fremont, CA (US); Ashley Thomas Baer, Glen Mills, PA (US); Paul J. Kettle, Red Wood City, CA (US)

(21) Appl. No.: 14/670,903

(22) Filed: Mar. 27, 2015

Related U.S. Application Data
(63) Continuation-in-part of application No. 14/463,699, filed on Aug. 20, 2014.
(60) Provisional application No. 62/096,585, filed on Dec. 24, 2014.
(60) Provisional application No. 61/871,934, filed on Aug. 30, 2013.

(51) Int. Cl. A61B 5/00; A61B 5/024; A61B 5/1455

(52) U.S. Cl. CPC A61B 5/7275; A61B 5/14551; A61B 5/02416; A61B 5/7203; A61B 5/6843

(57) ABSTRACT

A system includes a first light source configured to emit light within a first wavelength light band, and a second light source configured to emit light within a second wavelength light band, where the first wavelength light band and the second wavelength light band are each reflected differently by biologic material (e.g., blood). The system also includes a light sensor configured to detect light within the first wavelength light band and the second wavelength light band and generate a first signal representative of received light intensity within the first wavelength light band and a second signal representative of received light intensity within the second wavelength light band. The system further includes circuitry configured to correlate the first signal and the second signal to determine whether at least one of the first signal or the second signal is associated with a periodic biologic function.

### CROSS-REFERENCE TO RELATED APPLICATIONS

The present application claims the benefit under 35 U.S.C. §119(e) of U.S. Provisional Application Ser. No. 62/096,585, filed Dec. 24, 2014, and titled “SYSTEMS AND TECHNIQUES TO DETERMINE WHETHER A SIGNAL IS ASSOCIATED WITH A PERIOD BIOLOGIC FUNCTION.” The present application is also a continuation-in-part under 35 U.S.C. §120 of U.S. patent application Ser. No. 14/463,699, filed Aug. 20, 2014, and titled “DETECTING PRESSURE EXERTED ON A TOUCH SURFACE AND PROVIDING FEEDBACK,” which claims priority under 35 U.S.C. §119(e) of U.S. Provisional Application Serial No. 61/871,934, filed Aug. 30, 2013, and titled “DETECTING PRESSURE EXERTED ON A TOUCH SURFACE AND PROVIDING FEEDBACK.” U.S. patent application Ser. No. 14/463,699 and U.S. Provisional Application Ser. Nos. 61/871,934 and 62/096,585 are herein incorporated by reference in their entireties.

### BACKGROUND

A touch panel is a human machine interface (HMI) that allows an operator of an electronic device to provide input to the device using an instrument such as a finger, a stylus, and so forth. For example, the operator may use his or her fingers to manipulate images on an electronic display, such as a display attached to a mobile computing device, a personal computer (PC), or a terminal connected to a network. In some cases, the operator may use two or more fingers simultaneously to provide unique commands, such as a zoom command, executed by moving two fingers away from one another; a shrink command, executed by moving two fingers toward one another; and so forth. In other cases, the operator may use a stylus to provide commands via a touch panel.

A touch screen is an electronic visual display that incorporates a touch panel overlying a display to detect the presence and/or location of a touch within the display area of the screen. Touch screens are common in devices such as all-in-one computers, tablet computers, satellite navigation devices, gaming devices, and smartphones. A touch screen enables an operator to interact directly with information that is displayed by the display underlying the touch panel, rather than indirectly with a pointer controlled by a mouse or touchpad. Capacitive touch panels are often used with touch screen devices. A capacitive touch panel generally includes an insulator, such as glass, coated with a transparent conductor, such as indium tin oxide (ITO). As the human body is also an electrical conductor, touching the surface of the panel results in a distortion of the panel's electric field, measurable as a change in capacitance.

### SUMMARY

A system includes a first light source configured to emit light within a first wavelength light band, and a second light source configured to emit light within a second wavelength light band, where the first wavelength light band and the second wavelength light band are each reflected differently by biologic material. The system also includes a light sensor configured to detect light within the first wavelength light band and the second wavelength light band and generate a first signal representative of received light intensity within the first wavelength light band and a second signal representative of received light intensity within the second wavelength light band. The system further includes circuitry configured to correlate the first signal and the second signal to determine whether at least one of the first signal or the second signal is associated with a periodic biologic function.

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

### DRAWINGS

The Detailed Description is described with reference to the accompanying figures. The use of the same reference numbers in different instances in the description and the figures may indicate similar or identical items.

FIG. 1 is a partial diagrammatic illustration of a system that can be configured to initiate compensation for a touch pressure dependent characteristic detectable by the system, where the system can be configured to instruct a user to move a body part, such as a fingertip, to adjust the amount of pressure the user exerts on a touch surface and/or to modify a signal that measures the touch pressure dependent characteristic in accordance with an example embodiment of the present disclosure.

FIG. 2 is a diagrammatic illustration of circuitry for determining whether a signal, such as a signal from a pulse oximeter sensor device, is associated with a periodic biologic function, such as a heartbeat, in accordance with an example embodiment of the present disclosure.

FIG. 3 is a graph illustrating a signal generated by a red wavelength light band light-emitting diode and another signal generated by an infrared wavelength light band light-emitting diode of a pulse oximeter sensor device when appropriate pressure (e.g., appropriate finger pressure) is applied at a user's measuring site in accordance with an example embodiment of the present disclosure.

FIG. 4 is a graph illustrating a signal generated by a red wavelength light band light-emitting diode and another signal generated by an infrared wavelength light band light-emitting diode of a pulse oximeter sensor device when the system is at rest with the pulse oximeter sensor device placed a fixed distance from a non-biologic object (e.g., a table surface) in accordance with an example embodiment of the present disclosure.

FIG. 5 is a graph illustrating a signal generated by a red wavelength light band light-emitting diode and another signal generated by an infrared wavelength light band light-emitting diode of a pulse oximeter sensor device, where heart rate pulses are shown even when excessive pressure (e.g., excessive finger pressure) is applied in accordance with an example embodiment of the present disclosure.

FIG. 6 is a diagrammatic illustration of a system that can be configured to initiate compensation for a touch pressure dependent characteristic detectable by the system, where the system can be configured to instruct a user to move a body part, such as a fingertip, to adjust the amount of pressure the user exerts on a touch surface and/or to modify a signal that measures the touch pressure dependent characteristic, and where the system includes a camera in accordance with an example embodiment of the present disclosure.

FIG. 7 is a diagrammatic illustration of a system that can be configured to initiate compensation for a touch pressure dependent characteristic detectable by the system, where the system can be configured to instruct a user to move a body part, such as a fingertip, to adjust the amount of pressure the user exerts on a touch surface and/or to modify a signal that measures the touch pressure dependent characteristic, and where the system includes a touch screen in accordance with an example embodiment of the present disclosure.

FIG. 8 is a block diagram illustrating a system that can be configured to initiate compensation for a touch pressure dependent characteristic detectable by the system in accordance with an example embodiment of the present disclosure.

FIG. 9 is a flow diagram illustrating a method for determining whether a signal, such as a signal from a pulse oximeter sensor device, is associated with a periodic biologic function, such as a heartbeat, and initiating compensation for a touch pressure dependent characteristic in accordance with example embodiments of the present disclosure.

### DETAILED DESCRIPTION

Sensor devices that provide user monitoring functionality can be included in portable electronic devices, such as smartphones, portable health monitors, and so forth. These portable devices can be used to detect (e.g., measure) health and/or biological characteristics of a user, such as blood oxygen saturation, blood glucose concentration, and so on. Some sensor devices require a user to touch a substrate supporting a sensor in order to perform a detection operation. However, such sensor devices can be sensitive to pressure variations when the user touches the substrate. For example, a fingertip sensor device is calibrated to perform detection operations at a particular pressure exerted by the touch of the user's fingertip. When the user touches the substrate and exerts a different pressure, the results of the detection operation can be affected. For example, the results can be less accurate than when a desired level of pressure is exerted. Further, finger pressure can vary from person to person, and the signal strength received by a sensor can vary accordingly.

Additionally, when signals received by a sensor appear to have a poor (e.g., comparatively low) signal-to-noise ratio (SNR), an electronic device may not be able to determine whether the poor SNR is caused by undesirable (e.g., excessive) pressure exerted by a user, or by another condition, such as when the sensor is not in contact with flesh, e.g., when the electronic device is placed on a surface, such as a table surface. For example, circuitry for reflectance-based pulse oximetry equipment can have two channels, an infrared (IR) light channel and a visible red light channel. These two channels can each carry a direct current (DC) signal, and a small alternating current (AC) signal that includes bio-sensor information. However, the AC signal quality may depend on finger pressure applied on the pulse oximetry sensor. For instance, when excessive finger pressure is applied on the sensor, blood perfusion at a user's measuring site may be reduced, resulting in a poor SNR.

Systems and techniques are described that initiate compensation for a touch pressure dependent characteristic detectable by a system. In some embodiments, instructions are provided to a user when the user exerts pressure on a touch surface with a body part. For example, the systems determine finger pressure by correlating semi-independent and/or fully-independent optical bio-sensor channels, and instruct the user to move the finger to adjust the amount of pressure the user exerts on the touch surface. The instructions are configured to instruct the user to adjust the amount of pressure the user exerts on a bio-signal sensor, and can reduce pressure artifacts, increase the SNR ratio of bio-signal sensor output, and so forth. The systems can also modify a signal that measures the touch pressure dependent characteristic. In some embodiments, the systems comprise touch sensing systems, such as capacitive touch panels, touch screens, and so forth. In some embodiments, the systems comprise one or more cameras. As described herein, a system can be configured as a smart phone, a tablet computing device, a health monitor (e.g., a health monitor band), a fitness monitor (e.g., a fitness monitor band), and so forth. In example embodiments, the systems described herein can be used with smart phone cameras and/or touch screen devices.

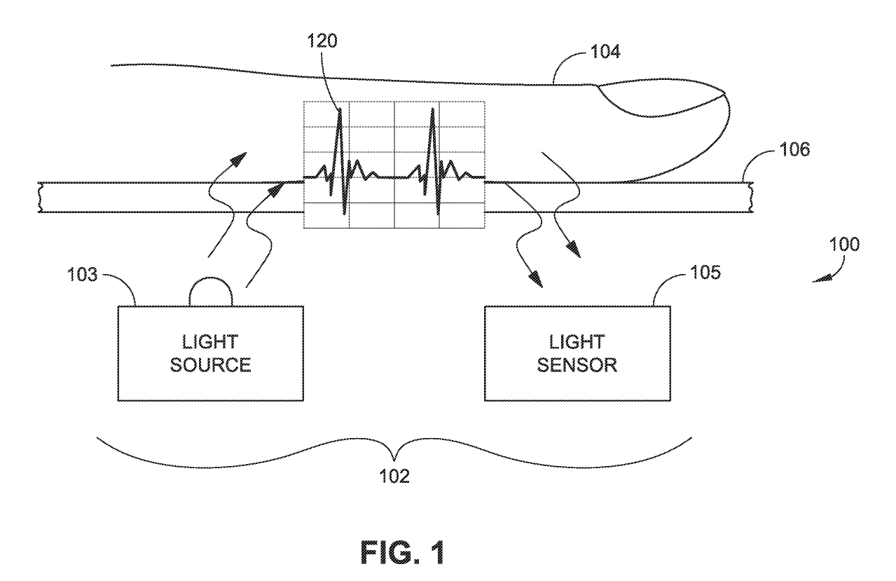

FIGS. 1, 2, and 6 through 8 illustrate example systems 100 in accordance with example implementations of the present disclosure. The systems 100 include a bio-signal sensor device (e.g., a pulse oximeter sensor device 102, a heart rate monitor device, and so forth) configured to detect a touch pressure dependent characteristic of a body part (e.g., a finger 104) of a user while the user touches a touch surface 106 with the body part. For example, the touch surface 106 comprises the housing of a smart phone, and the pulse oximeter sensor device 102 is configured as an integrated circuit (IC) chip comprising a light source 103 (e.g., a red wavelength light emitting diode (LED), an infrared (IR) LED, and so forth) and a light sensor 105 (e.g., a photodiode sensor) positioned under a glass touch surface 106. However, a smart phone is provided by way of example only and is not meant to limit the present disclosure. In other embodiments, the touch surface 106 is comprised of a tablet computing device, a health monitor (e.g., a health monitor band), a fitness monitor (e.g., a fitness monitor band), and so forth.

In some embodiments, the pulse oximeter sensor device 102 comprises a transmission pulse oximeter that transmits light through the finger 104. In other embodiments, the pulse oximeter sensor device 102 comprises a reflection pulse oximeter that reflects light from the finger 104. However, the pulse oximeter sensor device 102, the light source 103, and the light sensor 105 are provided by way of example only and are not meant to be restrictive of the present disclosure. In other embodiments, a bio-signal sensor can be configured to detect one or more of oxygen (O₂) saturation, a glucose concentration, a carbon monoxide (CO) concentration, a carbon dioxide (CO₂) concentration associated with blood in the body part of the user, and so forth. In some embodiments the bio-signal sensor can implement one or more sensor functionalities, including, but not necessarily limited to: a glucose sensor, a heart rate sensor (e.g., a heart rate monitor that uses a red LED, a green wavelength LED, an IR LED, another color wavelength emitting LED, a multi-color wavelength emitting LED, and so on, with one or more associated light sensors, and so forth).

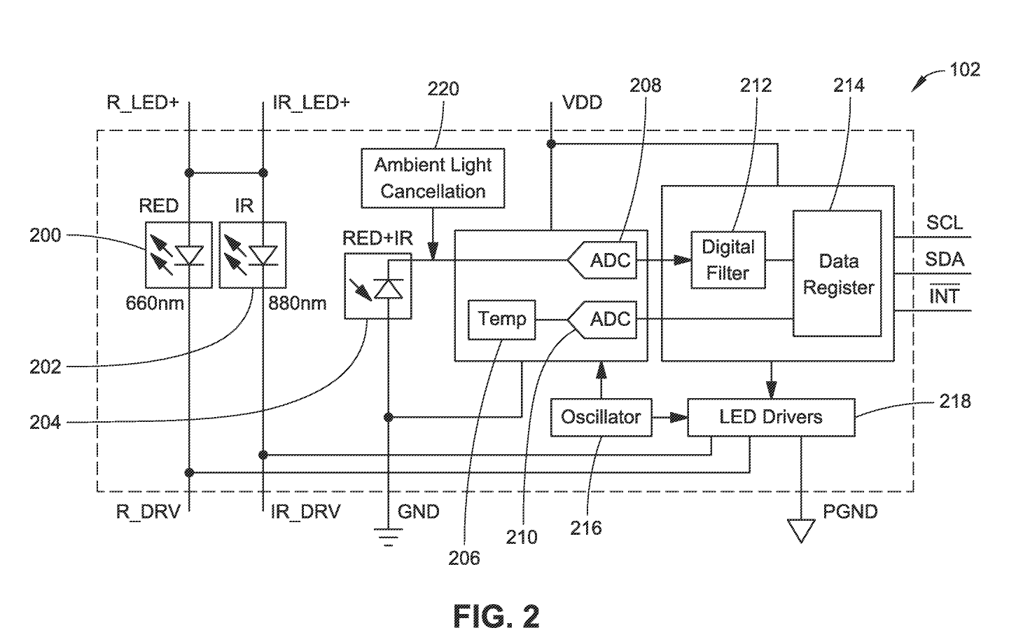

Referring now to FIG. 2, in embodiments of the disclosure, the pulse oximeter sensor device 102 includes a visible (e.g., red) light source, such as a light-emitting diode (LED) 200 operating in the red wavelength light band, between at least approximately six hundred nanometers (600 nm) and seven hundred and fifty nanometers (750 nm), e.g., at six hundred and sixty nanometers (660 nm). The pulse oximeter sensor device 102 also includes an infrared light source, such as an LED 202 operating in the infrared wavelength light band, between at least approximately eight hundred and fifty nanometers (850 nm) and one thousand nanometers (1,000 nm), e.g., at eight hundred and eighty nanometers (880 nm). In embodiments of the disclosure, the pulse oximeter sensor device 102 includes one or more light sensors, such as a photodiode 204 configured to detect light in the red and infrared wavelength light bands.

In some embodiments, the pulse oximeter sensor device 102 can use transmission-based techniques to determine (e.g., measure) one or more blood flow characteristics of a user. For example, the photodiode 204 can be positioned opposite the LED 200 and/or the LED 202. In this example, light can pass from the LED 200 and/or the LED 202, through the user's measuring site, and on to the photodiode 204, where red and/or infrared light is received by the photodiode 204. In other embodiments, the pulse oximeter sensor device 102 can use reflectance-based techniques to determine (e.g., measure) one or more blood flow characteristics of a user. For instance, the photodiode 204 can be positioned adjacent the LED 200 and/or the LED 202. In this example, light from the LED 200 and/or the LED 202 reflected from the user's measuring site can be received by the photodiode 204. In still further embodiments, the pulse oximeter sensor device 102 can use transmission and reflectance-based techniques to determine (e.g., measure) one or more blood flow characteristics of a user. For example, the pulse oximeter sensor device 102 can include a photodiode opposite the LED 200 and/or the LED 202, and another photodiode adjacent the LED 200 and/or the LED 202.

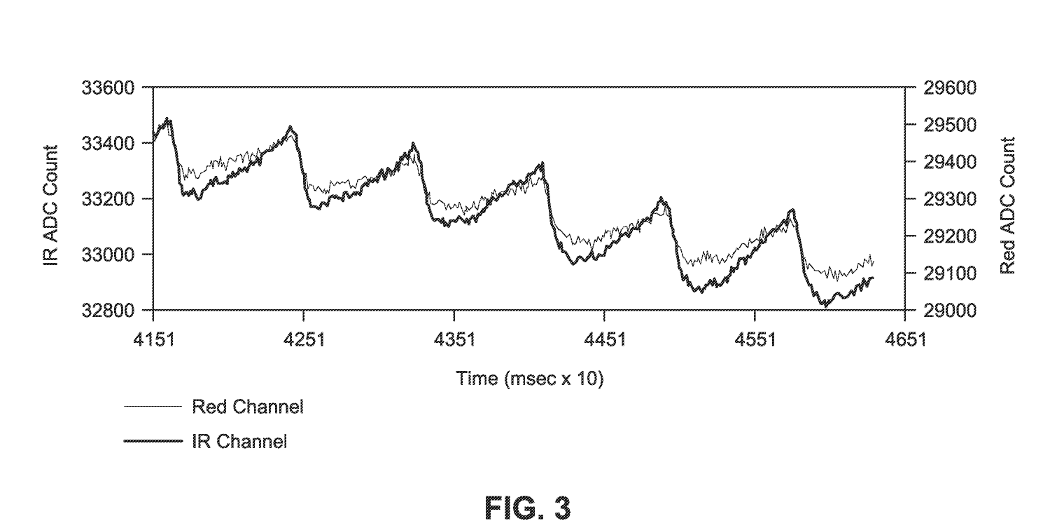

After red and infrared light is received at the photodiode 204, a red (R) to infrared ratio (R/IR) can be determined. The R/IR ratio can then be compared to data (e.g., empirical data) to determine one or more blood flow characteristics of a user. For example, oxygenated hemoglobin in blood absorbs more infrared light while allowing more red light to pass through the blood when compared to deoxygenated (reduced) hemoglobin. In some embodiments, an R/IR ratio determined for a user is compared to a lookup table comprising empirical formulas and converted to an oxygen saturation (e.g., SpO₂) value associated with the user. For example, a lookup table is compiled using a manufacturer's calibration curves (e.g., derived from healthy subjects at various SpO₂ levels). With each heartbeat, there will be a surge of arterial blood at the user's measuring site, momentarily increasing arterial blood volume across the site. Because of the light absorption properties of oxygenated hemoglobin, there will be more light absorption during each surge. Thus, light signals received by the photodetector 204 can be seen as a waveform, e.g., with peaks at each heartbeat and troughs between heartbeats (e.g., as shown in FIG. 3).

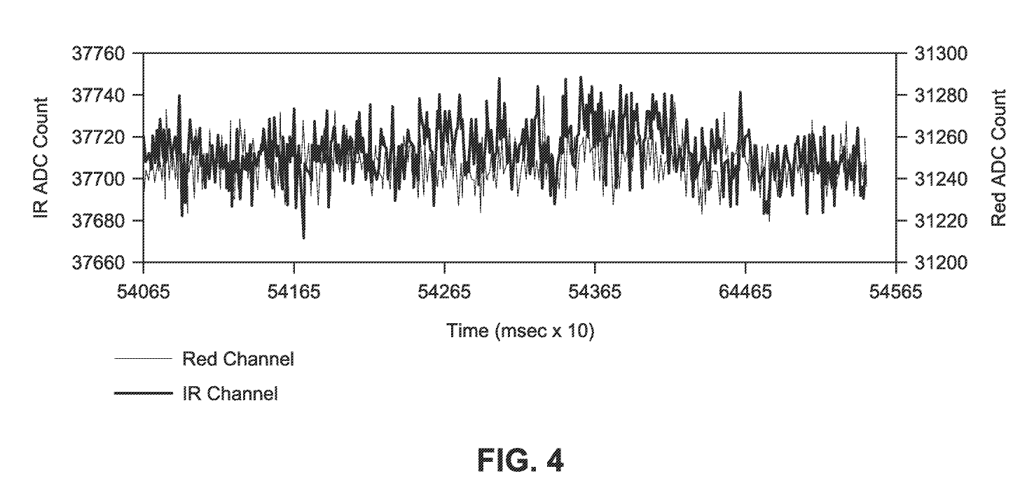

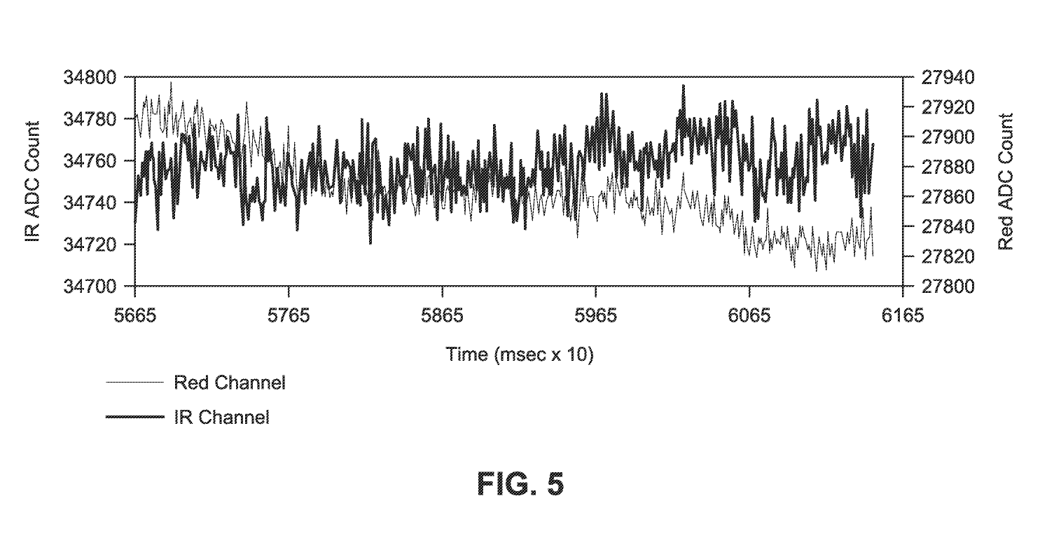

In embodiments of the disclosure, an optical bio-sensor, such as the pulse oximeter sensor device 102, can be used to determine whether a signal is associated with a periodic biologic function. For instance, the pulse oximeter sensor device 102 can be used to determine whether the sensor is applied to flesh or is unconnected (e.g., resting on a table). In some embodiments, the output of a pulse oximeter sensor device 102 can be no signal (e.g., a DC signal with noise) for the following cases: when the sensor is not in contact with flesh (e.g., as shown in FIG. 4), when excessive pressure is applied to the sensor (e.g., as shown in FIG. 5), and so forth. However, in the case of the pulse oximeter sensor device 102, even though signals from both the LED 200 and the LED 202 may have poor SNR in these scenarios, the noise can be correlated among both channels. This correlation can be used to identify if a poor SNR signal is caused by, for example, excessive finger pressure, or if the sensor is not in contact with flesh (e.g., positioned on a table). In this manner, correlation between semi-independent and/or fully-independent optical bio-sensor channels can be used to determine whether a sensor is connected to flesh (e.g., human flesh) or is not being used.

In example embodiments, a first DC signal representative of received light intensity within a first wavelength light band reflected from and/or transmitted through a biologic fluid (e.g., blood) can be generated (e.g., using the LED 200). A second DC signal representative of received light intensity within a second wavelength light band reflected from and/or transmitted through the biologic fluid can also be generated (e.g., using the LED 202). As described herein, the first wavelength light band and the second wavelength light band are each reflected differently by the biologic fluid. When the pulse oximeter sensor device 102 is connected to flesh, a bio-signal can be received as an AC signal riding on a DC signal. In some embodiments, the AC signal component of the received signal is comparatively smaller than the slow moving DC signal. The outputs of the two channels can be used to determine vital signals, including, but not necessarily limited to: heart rate, blood oxygen, respiration, and so forth.

In embodiments of the disclosure, too much finger pressure reduces blood perfusion, which can diminish the AC signal component of the received signal (e.g., leaving primarily a DC signal). Similar signals (e.g., no AC, high DC) can also be generated when a system 100 is at rest (e.g., where a light source is reflected from a resting surface and directly impacts the sensor area, resulting in a high DC signal). As described herein, the two DC signals can be correlated to determine whether a signal is associated with a periodic biologic function. For instance, the first DC signal from the LED 200 and the second DC signal from the LED 202 are correlated to determine whether the first DC signal and/or the second DC signal is associated with a heartbeat. As shown in FIG. 2, in some embodiments the pulse oximeter sensor device 102 can include circuitry comprising a temperature sensor 206, a first analog-to-digital converter (ADC) 208, a second ADC 210, a digital filter 212, a data register 214, an oscillator 216, LED drivers 218, circuitry for ambient light cancellation 220, and so forth. This circuitry can be used to correlate the DC signals from the LED 200 and the LED 202 to determine whether a signal is associated with a periodic biologic function, such as a heartbeat. However, it should be noted that the circuitry illustrated in FIG. 2 is provided by way of example and is not meant to limit the present disclosure. Thus, in other embodiments, different circuitry can be used to determine whether a signal is associated with a periodic biologic function.

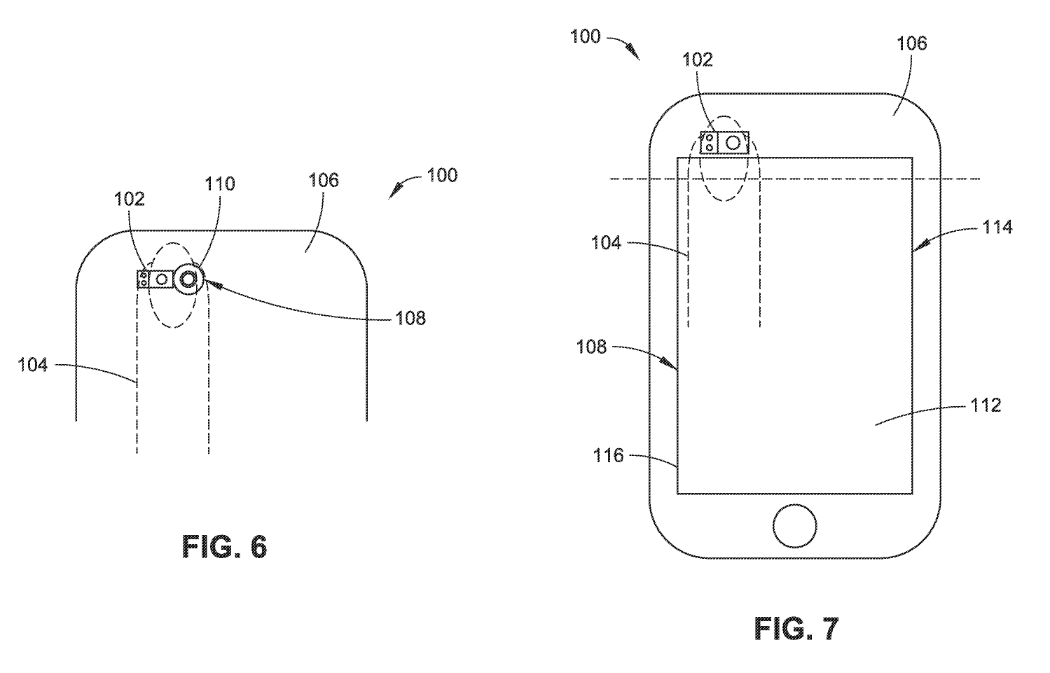

Referring now to FIG. 6, in some embodiments a system 100 can include an image capture device 108 configured to capture at least a partial image (e.g., a partial image, a full image, etc.) of the finger 104 of the user while the user touches the touch surface 106 with the finger 104. In some embodiments, the image capture device 108 comprises a camera 110 configured to detect light in the visible spectrum. In other embodiments, the camera 110 is configured to detect light in the IR spectrum (e.g., as reflected from the pulse oximeter sensor device 102). In still further embodiments, the camera 110 detects light in both the visible spectrum and the IR spectrum. In some embodiments, images captured by the camera are grayscale (e.g., shades of gray, black and white, etc.). In other embodiments, images captured by the camera are in color.

With reference to FIG. 7, in other embodiments, the image capture device 108 comprises a touch panel 112. For example, the systems 100 include one or more touch panels 112, such as mutual capacitance Projected Capacitive Touch (PCT) panels. The capacitive touch panels 112 are configured to sense multiple inputs simultaneously, or at least substantially simultaneously. The capacitive touch panels 112 can be included with electronic devices, including, but not necessarily limited to: large touch panel products, touchpad products, all-in-one computers, mobile computing devices (e.g., hand-held portable computers, Personal Digital Assistants (PDAs), laptop computers, netbook computers, tablet computers, and so forth), mobile telephone devices (e.g., cellular telephones and smartphones), devices that include functionalities associated with smartphones and tablet computers (e.g., phablets), portable game devices, portable media players, multimedia devices, satellite navigation devices (e.g., Global Positioning System (GPS) navigation devices), e-book reader devices (eReaders), Smart Television (TV) devices, surface computing devices (e.g., table top computers), Personal Computer (PC) devices, as well as with other devices that employ touch-based human interfaces.

The capacitive touch panels 112 can comprise ITO touch panels that include drive electrodes, such as X-axis and/or Y-axis cross-bar ITO drive traces/tracks, arranged next to one another (e.g., along parallel tracks, generally parallel tracks, and so forth). The drive electrodes are elongated (e.g., extending along a longitudinal axis). For example, each drive electrode extends along an axis on a supporting surface, such as a substrate of a capacitive touch panel 112. The capacitive touch panels 112 also include sense electrodes, such as cross-bar X-axis and/or Y-axis ITO sensor traces/tracks, arranged next to one another across the drive electrodes (e.g., along parallel tracks, generally parallel tracks, and so forth). The sense electrodes are elongated (e.g., extending along a longitudinal axis). For instance, each sense electrode extends along an axis on a supporting surface, such as a substrate of a capacitive touch panel 112. It should be noted that an ITO touch panel 112 is provided by way of example only and is not meant to limit the present disclosure. In other embodiments, one or more other transparent materials (e.g., Antimony Tin Oxide (ATO)), semi-transparent materials, and/or non-transparent materials (e.g., copper) is used for a drive electrode and/or a sense electrode of a capacitive touch panel.

The drive electrodes and the sense electrodes define a coordinate system where each coordinate location (pixel) comprises a capacitor formed at each junction between one of the drive electrodes and one of the sense electrodes. Thus, the drive electrodes are configured to connect to one or more electrical circuits and/or electronic components (e.g., one or more drivers) to generate a local electric field at each capacitor. A change in the local electric field generated by an instrument (e.g., input from a finger or a stylus) at each capacitor formed at a drive electrode and a sense electrode causes a change (e.g., a decrease) in capacitance associated with a touch at the corresponding coordinate location. Mutual capacitance is capacitance that occurs between two charge-holding objects (e.g., conductors). In this instance, mutual capacitance is the capacitance between the drive electrodes and the sense electrodes that comprise the capacitive touch panel sensor. As described above, the drive electrodes and the sense electrodes comprise traces that represent the driving lines and corresponding sensing lines to detect a change in mutual capacitance due to a touch event performed over the surface of the touch panel 112. It should be noted that for the purposes of the present disclosure, the drive electrodes comprise the driving lines and the sense electrodes comprise the sensing lines in some implementations, and the drive electrodes comprise the sensing lines and the sense electrodes comprise the driving lines in other implementations.

It should also be noted that capacitive touch panels 112 as described herein are not limited to mutual capacitance sensing. For example, input from a finger can also be sensed via self capacitance of one or more of the capacitive touch panel sensors. Self capacitance is the capacitance associated with the respective column and the respective row and represents the amount of electrical charge to be furnished to the respective column or row to raise its electrical potential by one unit (e.g., by one volt, and so on). In embodiments of the disclosure, more than one touch can be sensed at differing coordinate locations simultaneously (or at least substantially simultaneously). In some embodiments, the drive electrodes are driven by one or more of the drivers in parallel, e.g., where a set of different signals are provided to the drive electrodes. In other embodiments, the drive electrodes are driven by one or more of the drivers in series, e.g., where each drive electrode or subset of drive electrodes is driven one at a time.

The sense electrodes are electrically insulated from the drive electrodes (e.g., using a dielectric layer, and so forth). For example, the sense electrodes are provided on one substrate (e.g., comprising a sense layer disposed on a glass substrate), and the drive electrodes are provided on a separate substrate (e.g., comprising a drive layer disposed on another substrate). In this two-layer configuration, the sense layer can be disposed above the drive layer (e.g., with respect to a touch surface). For example, the sense layer is positioned closer to a touch surface than the drive layer. However, this configuration is provided by way of example only and is not meant to be restrictive of the present disclosure. Thus, other configurations can be provided where the drive layer is positioned closer to a touch surface than the sense layer, and/or where the sense layer and the drive layer comprise the same layer. For instance, in a 1.5-layer embodiment (e.g., where the drive layer and the sense layer are included on the same layer but physically separated from one another), one or more jumpers are used to connect portions of a drive electrode together. Similarly, jumpers can be used to connect portions of a sense electrode together. In other embodiments, the drive layer and the sense layer comprise the same layer (e.g., in a single-layer sensor configuration).

One or more capacitive touch panels 112 can be included with a touch screen assembly 114. The touch screen assembly 114 includes a display screen 116, such as a liquid crystal display (LCD) screen, where the sense layer and the drive layer are sandwiched between the LCD screen and a bonding layer, with a protective cover (e.g., cover glass) attached thereto. The cover glass can include a protective coating, an anti-reflective coating, and so forth. The cover glass comprises a touch surface, upon which an operator can use one or more fingers, a stylus, and so forth to input commands to the touch screen assembly 114. The commands can be used to manipulate graphics displayed by, for example, the LCD screen. Further, the commands can be used as input to an electronic device connected to a capacitive touch panel 112, such as a multimedia device or another electronic device (e.g., as previously described).

The system 100 can be configured to compare the amount of pressure exerted on the touch surface 106 by the user to a desired amount of pressure associated with the user for the bio-signal sensor device. For example, a desired amount of pressure can be determined for a particular user or a group of users by correlating semi-independent and/or fully-independent optical bio-sensor channels. This calibration can be performed using an amount of pressure exerted on the touch surface 106 by a user when initial bio-signals are collected from the user (e.g., upon device startup, device initialization, and so forth). In some embodiments, these bio-signals are compared with bio-signals measured with another bio-sensor simultaneously, or substantially simultaneously. In other embodiments, bio-signals collected using the pulse oximeter sensor device 102 are compared to known (e.g., baseline) bio-signal information for the user. These comparisons are then used to determine a desired amount of pressure to be exerted by the user (e.g., a pressure that generates bio-signals within a desired range, having a certain degree of accuracy, and so forth). Further, such calibration can be performed for a particular size and/or range of sizes for a body part (e.g., finger size), a particular biological characteristic (e.g., profusion rate), and so forth.

In some embodiments, the system 100 is configured to initiate an instruction to the user to move the finger 104 to adjust the amount of pressure exerted on the touch surface 106 toward the desired amount of pressure. For example, the system 100 is configured to instruct the user to move the finger 104 toward the desired amount of pressure associated with the user for the pulse oximeter sensor device 102. Accordingly, the system 100 includes an indicator 118 configured to provide instructions to move the finger 104. In some embodiments, the indicator 118 comprises the touch screen assembly 114. For instance, graphical instructions (e.g., directional arrows) can be provided that instruct the user to move the finger 104. However, graphical instructions are provided by way of example only and are not meant to limit the present disclosure. In other embodiments, the indicator 118 can provide audio instructions, tactile instructions, haptic feedback, and so on to instruct the user. For the purposes of the present disclosure, adjusting pressure exerted on the touch surface 106 includes increasing or decreasing an amount of surface area of a body part in contact with the touch surface 106, making a positional adjustment (e.g., finger placement) of a body part with respect to the touch surface 106, and so forth. For example, the instructions can be used to achieve consistent placement of the finger 104.

In some embodiments, the system 100 also includes a gyroscope, an accelerometer, and so forth, which can be used to reduce motion artifacts when sensing bio-signals. Further, the system 100 can be configured to adjust the current through a bio-signal sensor, such as the pulse oximeter sensor device 102, based upon a detected amount of pressure exerted by a user. For example, more current can be supplied based upon a detected pressure level to increase the SNR of the system 100 when bio-signals are detected. In some embodiments, current through the pulse oximeter sensor device 102 is adjusted in discrete steps. In other embodiments, the current is adjusted continuously, based upon values in a lookup table, and so forth. Further, it will be appreciated that the touch surface 106 and the image capture device 108 are not necessarily disposed on the same side of the housing of an electronic device, such as a smart phone. For example, the image capture device 108 and the pulse oximeter sensor device 102 are disposed on opposite sides (e.g., front and back sides) of a smart phone. Further, in some embodiments, another sensor is used to augment the pressure detecting capability of the system 100. For example, the system 100 includes a pressure sensor comprising an aperture defined in the touch surface 106 for sensing barometric pressure, where blockage of the aperture is associated with pressure exerted on the touch surface 106.

In some embodiments, a signal 120 is modified that measures the touch pressure dependent characteristic of the body part (e.g., the finger 104) of the user. For example, touch and/or pressure information detected by the system 100 can be used with one or more motion compensation algorithms that compensate for a touch pressure dependent characteristic (e.g., in a case where the user is rolling finger 104 across the touch surface 106, which may be detectable by a change in the capacitive image). In some embodiments, modifying the signal 120 comprises discarding data comprising a portion of the signal 120. In other embodiments, modifying the signal 120 comprises removing one or more artifacts from the signal 120. For example, when the signal 120 is undetectable and strong finger pressure is detected, updates are not necessarily provided. In this example, instructions are provided to the user to apply less pressure. When the signal 120 is weak but still detectable, then an attempt to use the data can be made, along with feedback to the user that the signal could be improved by applying less pressure. In another example, when a sudden change in finger pressure is detected, the data can be discarded during the abrupt change, and updating can resume when the artifact subsides, and/or an attempt can be made to compensate for the change in pressure.

In a further example, when finger rolling is detected, the data can be discarded, and the output can cease, and/or an attempt can be made to remove the artifact (e.g., using a motion compensation algorithm). In this example, feedback can be provided to the user stating that the finger 104 should be kept still. In another example, when an incorrect finger position is detected (e.g., using the camera 110), feedback can be provided to the user. In this example, if a weak signal is still present, an attempt can be made to use the signal; otherwise, output is not necessarily provided. In a still further example, when removal of the finger 104 is detected (e.g., using the camera 110), the data can be temporarily discarded, and updating can resume when the finger 104 is replaced. However, if the finger 104 is not replaced quickly, a reset operation may be performed while waiting for the signal 120 to resume. In another example, when motion consistent with walking motion and/or running motion of the user is detected (e.g., a harmonic change in pressure), an attempt can be made to remove artifacts from the signal (e.g., using a motion compensation algorithm).

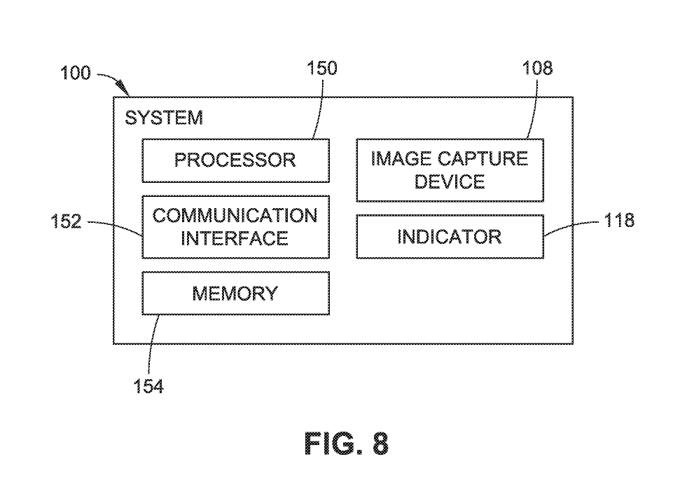

Referring now to FIG. 8, a system 100, including some or all of its components, can operate under computer control. For example, a processor 150 can be included with or in a system 100 to control the components and functions of systems 100 described herein using software, firmware, hardware (e.g., fixed logic circuitry), manual processing, or a combination thereof. The terms “controller,” “functionality,” “service,” and “logic” as used herein generally represent software, firmware, hardware, or a combination of software, firmware, or hardware in conjunction with controlling the systems 100. In the case of a software implementation, the module, functionality, or logic represents program code that performs specified tasks when executed on a processor (e.g., central processing unit (CPU) or CPUs). The program code can be stored in one or more computer-readable memory devices (e.g., internal memory and/or one or more tangible media), and so on. The structures, functions, approaches, and techniques described herein can be implemented on a variety of commercial computing platforms having a variety of processors.

As described, the system 100 includes a processor 150, a communications interface 152, and a memory 154. The processor 150 provides processing functionality for the system 100 and can include any number of processors, micro-controllers, or other processing systems and resident or external memory for storing data and other information accessed or generated by the system 100. The processor 150 can execute one or more software programs, which implement techniques described herein. The processor 150 is not limited by the materials from which it is formed or the processing mechanisms employed therein, and as such, can be implemented via semiconductor(s) and/or transistors (e.g., using electronic Integrated Circuit (IC) components), and so forth. The communications interface 152 is operatively configured to communicate with components of the touch panel. For example, the communications interface 152 can be configured to control the drive electrodes and/or the sense electrodes of the touch panel, receive inputs from the sense electrodes and/or the drive electrodes of the touch panel, and so forth. The communications interface 152 is also communicatively coupled with the processor 150 (e.g., for communicating inputs from the sense electrodes of the capacitive touch panel to the processor 150).

The communications interface 152 and/or the processor 150 can be configured to communicate with a variety of different networks, including, but not necessarily limited to: a wide-area cellular telephone network, such as a 3G cellular network, a 4G cellular network, or a global system for mobile communications (GSM) network; a wireless computer communications network, such as a WiFi network (e.g., a wireless local area network (WLAN) operated using IEEE 802.11 network standards); an internet; the Internet; a wide area network (WAN); a local area network (LAN); a personal area network (PAN) (e.g., a wireless personal area network (WPAN) operated using IEEE 802.15 network standards); a public telephone network; an extranet; an intranet; and so on. However, these networks are provided by way of example only and are not meant to limit the present disclosure. Further, the communications interface 152 can be configured to communicate with a single network or multiple networks across different access points.

The memory 154 is an example of tangible computer-readable media that provides storage functionality to store various data associated with operation of the system 100, such as software programs and/or code segments, or other data to instruct the processor 150 and possibly other components of the system 100 to perform the steps described herein. Thus, the memory 154 can store data, such as a program of instructions for operating the system 100 (including its components), and so forth. It should be noted that while a single memory 154 is shown, a wide variety of types and combinations of memory can be employed. The memory 154 can be integral with the processor 150, can comprise stand-alone memory, or can be a combination of both. The memory 154 can include, but is not necessarily limited to: removable and non-removable memory components, such as Random Access Memory (RAM), Read-Only Memory (ROM), Flash memory (e.g., a Secure Digital (SD) memory card, a mini-SD memory card, and/or a micro-SD memory card), magnetic memory, optical memory, Universal Serial Bus (USB) memory devices, and so forth. In embodiments, the system 100 and/or the memory 154 can include removable Integrated Circuit Card (ICC) memory, such as memory provided by a Subscriber Identity Module (SIM) card, a Universal Subscriber Identity Module (USIM) card, a Universal Integrated Circuit Card (UICC), and so on.

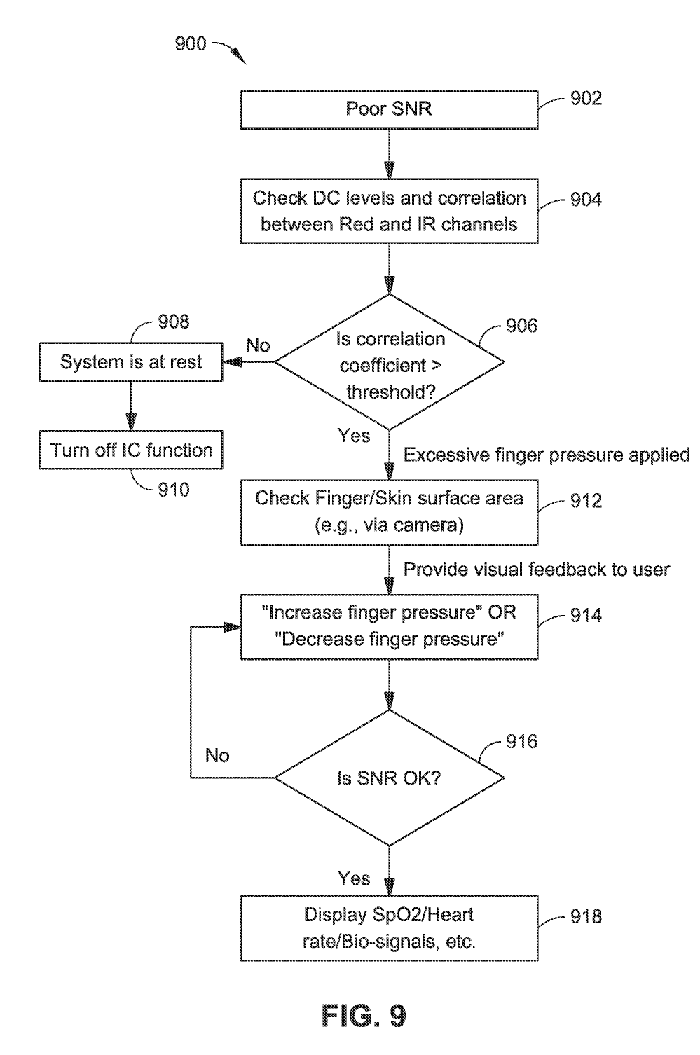

The following discussion describes example techniques for determining whether a signal is associated with a periodic biologic function and initiating compensation for a touch pressure dependent characteristic. FIG. 9 depicts a procedure 900, in example embodiments, in which it is determined whether a signal from a pulse oximeter sensor device is associated with a periodic biologic function, such as a heartbeat, and instructions can be initiated to a user to instruct the user to move a body part, such as a fingertip, to adjust the amount of pressure the user exerts on a touch surface, and/or a signal that measures the touch pressure dependent characteristic can be modified. In the procedure 900 illustrated, a signal with poor SNR is received (Block 902). For example, with reference to FIG. 1, a signal is received from the pulse oximeter sensor device 102, which is positioned under touch surface 106 and used to measure oxygen saturation for a user when the user touches the touch surface 106 with finger 104. Then, DC levels are checked between two channels, such as a red wavelength light band channel and an infrared wavelength light band channel, and a correlation between the two channels is performed (Block 904). For instance, with reference to FIG. 2, signals from the LED 200 and the LED 202 are correlated.

Next, a correlation coefficient can be compared to a threshold (Decision Block 906). If the correlation coefficient is less than the threshold, a determination can be made that the system is at rest (Block 908), and the function of the sensor can be turned off (Block 910). For example, if it is determined that the system 100 is at rest, the pulse oximeter sensor device 102 can be powered down. However, if the correlation coefficient is greater than the threshold, a determination can be made that excessive pressure (e.g., excessive finger pressure) is applied to the sensor. In some embodiments, an amount of finger and/or skin surface area covering the sensor can then be determined (Block 912). For instance, with reference to FIGS. 6 and 7, image capture device 108 captures an image (e.g., a partial image, a full image, etc.) of the finger 104 while the user touches the touch surface 106 with the finger 104. In some embodiments, the at least partial image is associated with an amount of pressure exerted on the touch surface by the user. For example, processor 150 associates the image captured by the image capture device 108 with an amount of pressure exerted on the touch surface 106 by determining an area of the touch surface 106 pressed by the finger 104. Further, the amount of pressure exerted on the touch surface by the user can be compared to a desired amount of pressure associated with the user for the sensor device. For instance, the correlation coefficient is compared to another correlation coefficient determined for the user's finger using a calibration operation.

Next, compensation is initiated for the touch pressure dependent characteristic based upon the comparison (Block 914). In some embodiments, an instruction is initiated to move the body part to adjust the amount of pressure exerted on the touch surface by the user toward the desired amount of pressure associated with the user for the sensor device. For example, with reference to FIGS. 7 and 8, indicator 118 (e.g., touch screen assembly 114) provides graphical instructions, such as directional arrows, that instruct the user to move the finger 104. In some embodiments, a signal is modified that measures the touch pressure dependent characteristic. In some embodiments, modifying the signal that measures the touch pressure dependent characteristic comprises discarding data comprising a portion of the signal. In other embodiments, modifying the signal that measures the touch pressure dependent characteristic comprises removing an artifact from the signal (e.g., as previously described). Then, the SNR of the resulting signal is checked (Decision Block 916). If the SNR of the signal is still too low, compensation can again be initiated for the touch pressure dependent characteristic based upon the comparison (Block 914). Otherwise, vital signals including heart rate, blood oxygen, respiration, and so forth can be displayed (Block 918).

Generally, any of the functions described herein can be implemented using hardware (e.g., fixed logic circuitry such as integrated circuits), software, firmware, manual processing, or a combination thereof. Thus, the blocks discussed in the above disclosure generally represent hardware (e.g., fixed logic circuitry such as integrated circuits), software, firmware, or a combination thereof. In the instance of a hardware configuration, the various blocks discussed in the above disclosure may be implemented as integrated circuits along with other functionality. Such integrated circuits may include all of the functions of a given block, system, or circuit, or a portion of the functions of the block, system, or circuit. Further, elements of the blocks, systems, or circuits may be implemented across multiple integrated circuits. Such integrated circuits may comprise various integrated circuits, including, but not necessarily limited to: a monolithic integrated circuit, a flip chip integrated circuit, a multichip module integrated circuit, and/or a mixed signal integrated circuit. In the instance of a software implementation, the various blocks discussed in the above disclosure represent executable instructions (e.g., program code) that perform specified tasks when executed on a processor. These executable instructions can be stored in one or more tangible computer readable media. In some such instances, the entire system, block, or circuit may be implemented using its software or firmware equivalent. In other instances, one part of a given system, block, or circuit may be implemented in software or firmware, while other parts are implemented in hardware.

Although the subject matter has been described in language specific to structural features and/or process operations, it is to be understood that the subject matter defined in the appended claims is not necessarily limited to the specific features or acts described above. Rather, the specific features and acts described above are disclosed as example forms of implementing the claims.

What is claimed is:

1. A system for determining whether a signal is associated with a periodic biologic function, the system comprising:
a first light source configured to emit light within a first wavelength light band;
a second light source configured to emit light within a second wavelength light band, the first wavelength light band and the second wavelength light band each reflected differently by oxygenated hemoglobin and reduced hemoglobin;
a light sensor configured to detect light within the first wavelength light band and the second wavelength light band and generate a first signal representative of received light intensity within the first wavelength light band and a second signal representative of received light intensity within the second wavelength light band; and
circuitry configured to correlate the first signal and the second signal to determine whether at least one of the first signal or the second signal is associated with the periodic biologic function.

2. The system as recited in claim 1, wherein the periodic biologic function comprises a heartbeat.

3. The system as recited in claim 1, wherein the first wavelength light band comprises visible red light and the second wavelength light band comprises infrared light.

4. The system as recited in claim 1, further comprising an image capture device configured to capture an at least partial image of a body part of a user while the user touches a touch surface with the body part, the body part comprising the oxygenated hemoglobin and the reduced hemoglobin.

5. The system as recited in claim 1, further comprising an indicator configured to provide an instruction to move a body part to a user, the body part comprising the oxygenated hemoglobin and the reduced hemoglobin.

6. The system as recited in claim 5, wherein the instruction comprises an instruction to move the body part to adjust an amount of pressure exerted on a touch surface by the user toward a desired amount of pressure associated with the user.

7. The system as recited in claim 1, wherein the circuitry is configured to initiate compensation for at least one of the first signal or the second signal.

8. The system as recited in claim 7, wherein initiating compensation for at least one of the first signal or the second signal comprises discarding data comprising at least a portion of at least one of the first signal or the second signal.

9. The system as recited in claim 7, wherein initiating compensation for at least one of the first signal or the second signal comprises removing an artifact from at least one of the first signal or the second signal.

10. A method for determining whether a signal is associated with a periodic biologic function, the method comprising:
generating a first direct current (DC) signal representative of received light intensity within a first wavelength light band at least one of reflected from or transmitted through a biologic material;
generating a second DC signal representative of received light intensity within a second wavelength light band at least one of reflected from or transmitted through the biologic material, the first wavelength light band and the second wavelength light band each reflected differently by the biologic material; and
correlating the first DC signal and the second DC signal to determine whether at least one of the first DC signal or the second DC signal is associated with the periodic biologic function.

11. The method as recited in claim 10, wherein the biologic material comprises oxygenated hemoglobin and reduced hemoglobin, and the periodic biologic function comprises a heartbeat.

12. The method as recited in claim 10, wherein the first wavelength light band comprises visible red light and the second wavelength light band comprises infrared light.

13. The method as recited in claim 10, further comprising capturing an at least partial image of a body part of a user while the user touches a touch surface with the body part, the body part comprising the biologic material.

14. The method as recited in claim 10, further comprising initiating compensation for at least one of the first signal or the second signal.

15. The method as recited in claim 14, wherein initiating compensation for at least one of the first signal or the second signal comprises initiating an instruction to move a body part to a user, the body part comprising the biologic material.

16. The method as recited in claim 15, wherein the instruction comprises an instruction to move the body part to adjust an amount of pressure exerted on a touch surface by the user toward a desired amount of pressure associated with the user.

17. The method as recited in claim 14, wherein initiating compensation for at least one of the first signal or the second signal comprises discarding data comprising at least a portion of at least one of the first signal or the second signal.

18. The method as recited in claim 14, wherein initiating compensation for at least one of the first signal or the second signal comprises removing an artifact from at least one of the first signal or the second signal.

19. A system for determining whether a signal is associated with a heartbeat, the system comprising:
a first light source configured to emit light within a visible red light band;
a second light source configured to emit light within an infrared light band;
a light sensor configured to detect light within the visible red light band and the infrared light band and generate a first signal representative of received light intensity within the visible red light band and a second signal representative of received light intensity within the infrared light band; and
circuitry configured to correlate the first signal and the second signal to determine whether at least one of the first signal or the second signal is associated with the heartbeat.

20. The system as recited in claim 19, wherein the circuitry is configured to initiate compensation for at least one of the first signal or the second signal.
